(19) United States
(12) Patent Application Publication
(10) Pub. No.: US 2010/0143424 A1
(43) Pub. Date: Jun. 10, 2010

(54) CASEIN NANOPARTICLE

(75) Inventor: Katsuhiko KANAZAWA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/630,575

(22) Filed: Dec. 3, 2009

(30) Foreign Application Priority Data
Dec. 5, 2008 (JP) 2008-310530

(51) Int. Cl. C07K 14/435; B32B 5/16; A61K 8/02; A61K 9/50; A23J 1/20; A61Q 17/04; C11D 17/00; A23L 1/302; A23L 1/305; A61K 31/7088; A61K 38/02; A61K 39/00; A61Q 19/10; A61P 25/28; A61Q 17/00; A61P 17/00; A61P 29/00; A61P 25/22; A61P 11/00; A61P 9/00; A61P 9/06; A61P 25/24

(52) U.S. Cl. 424/401; 530/360; 428/402; 424/491; 426/657; 424/59; 510/130; 426/648; 426/655; 514/44 R; 514/2; 424/184.1; 977/773; 977/898; 977/906; 977/962

(57) ABSTRACT

It is an object of the present invention to provide: a positively charged nanoparticle which can be produced without using surfactants or synthetic polymers, the size of which can be controlled, which is stable at acidic condition, and which contains an active substance therein; and a method for producing the same. The present invention provides a casein particle, wherein zeta potential is positive.

### TECHNICAL FIELD

The present invention relates to a nanoparticle for use in fields such as life science or medical diagnosis. More specifically, the present invention relates to a casein nanoparticle.

### BACKGROUND ART

Fine particle materials have been expected to be widely used in biotechnology. Particularly, studies have been conducted actively in recent years on the application of nanoparticle materials developed by the advances of nanotechnology to biotechnology or medical care. Many study results have been reported.

Nanoparticles have been expected strongly from early in the field of a drug delivery system (DDS) and are exceedingly promising as a carrier for drugs or genes. Particularly, studies using polymer micelle have been conducted actively. In most cases, AB- or ABA-type block copolymers are used because of the simplicity of their structures. The polymer micelle is characterized by its large drug capacity, high water solubility, high structural stability, non-accumulation, small particle size (100 nm or smaller), and functional separation. From this viewpoint, studies intended for targeting to target sites and solubilization of hydrophobic drugs have been conducted.

In recent years, cosmetics have embodied various novel techniques including nanotechnology and have thereby achieved improvement in functionality and usability and differentiation from other companies' products. More distinct effects on the skin have been demanded for cosmetics. Since the skin generally contains the stratum corneum as a barrier, the skin infiltration property of a drug is low. Improvement in the skin permeability of an active ingredient is essential for exerting sufficient effects on the skin. Many ingredients, even if having high effectiveness to the skin, are difficult to make into preparations because they have poor storage stability and are apt to cause skin irritation. The development of various capsules intended for improvement in percutaneous absorbability and storage stability, reduction in skin irritation, and so on have been pursued to solve these problems. Studies have been conducted currently on ultrafine emulsification or a variety of raw materials such as liposomes (e.g., M. Nishida, Fragrance Journal, the November issue, 17 (2005)). However, surfactants used in emulsification raise safety concerns. Moreover, structure formation with ion complexes produces poor stability as compared with that with covalent bond.

The use of polymer materials is expected to considerably improve storage stability and in-vivo particle stability. However, most studies use synthetic polymers produced by emulsion polymerization or the like. Although toxicity is reduced in the synthetic polymers as compared with low-molecular substances, toxicity to some extent should be expected. Therefore, a safer carrier has been demanded.

Natural polymers exhibit high structural stability as with synthetic polymers and have safety much higher than that of synthetic polymers. Thus, the natural polymers have both advantages as a DDS carrier. However, a difficult point of the natural polymer carrier as compared with synthetic polymers is a method for producing particles. Spray drying, freeze drying, and jet milling can be utilized as methods for producing natural polymer particles. However, in most cases, the particle size is a micron size and is difficult to control.

Among such natural polymers, casein is a water-insoluble protein contained in milk. Since its hydrophobic portion is exposed to the outside environment, casein is likely to form aggregates. 10 to 100 casein molecules gather and form a submicelle of approximately 20 nm. Furthermore, 100 to 1000 casein molecules gather and form a casein micelle of 90 to 150 nm. The casein micelles further gather and form a micelle associate of approximately 500 nm.

A casein micelle has a wide size distribution, and it is aggregated when supplemented with a salt of sodium, potassium, or the like or placed at an acidic pH. In the conventional methods for producing casein submicelles, casein submicelles have been produced using a basic solution of pH 6.5 or more. Hence, if the pH is decreased to a pH value lower than the isoelectric point of casein, although it is an amphiphilic protein, precipitation and aggregation occur. Thus, the state of such casein submicelles can not be maintained, and nanoparticles with positive charge can not be produced, and as a result, an anionic compound could not be incorporated into the nanoparticle or could not be retained on the particle surface (Journal of the agricultural chemical society of Japan, Vol. 16, No. 9, 1087-1092 (1987), Journal of the agricultural chemical society of Japan, Vol. 49, No. 8, 417-424 (1975), Journal of Dairy Research, 53, 547-555 (1986), and International Publication WO2007/114262 (FUJIFILM).

In JP Patent Publication (Kokai) No. 5-146258 A (1993), casein was exceptionally dissolved in an acidic solution. However, this was intended to fractionate the casein, and thus it was not intended to the use of submicelles.

### DISCLOSURE OF THE INVENTION

It is an object of the present invention to solve the aforementioned problems of the prior art techniques. That is to say, it is an object of the present invention to provide: a positively charged nanoparticle which can be produced without using surfactants or synthetic polymers, the size of which can be controlled, which is stable at acidic condition, and which contains an active substance therein; and a method for producing the same.

As a result of intensive studies directed towards achieving the aforementioned objects, the present inventors have found that a casein particle wherein zeta potential is positive can be produced by adding casein to an acidic solution, and then increasing the pH of the solution to a pH value that is ±pH 0.5 or more apart from the isoelectric point of the casein, thereby completing the present invention.

The present invention provides a casein particle, wherein zeta potential is positive.

Preferably, the casein particle has a mean particle diameter from 10 nm or more to less than 300 nm, in terms of particle size.

The present invention further provides a casein particle having a mean particle diameter from 10 nm or more to less than 300 nm, which is produced by the following steps (a) and (b):

(a) a step of mixing casein into an acidic aqueous medium from pH 0.5 or more to less than pH 7; and
(b) a step of increasing the pH of the solution obtained in the step (a) above to a pH value that is ±pH 0.5 or more apart from the isoelectric point of the casein, while stirring the solution.

Preferably, the casein particle according to the present invention comprises an active substance in the particle thereof and/or on the particle surface thereof.

Preferably, the active substance is an ionic substance or a fat-soluble substance.

Preferably, the active substance is an anionic substance.

The present invention further provides the casein particle according to the present invention which is produced by the following steps (a), (b), and (c):

(a) a step of mixing casein into an acidic aqueous medium from pH 0.5 or more to less than pH 7;
(b) a step of increasing the pH of the solution obtained in the step (a) above to a pH value that is ±pH 0.5 or more apart from the isoelectric point of the casein, while stirring the solution; and
(c) a step of adding at least one type of active substance to the solution obtained in the step (a) or (b) above.

Preferably, the active substance is a cosmetic ingredient, a functional food ingredient, or a pharmaceutical ingredient.

Preferably, the cosmetic ingredient is a moisturizer, a skin-lightening agent, a hair growth stimulant, a hair restorer, a hair growing agent, an anti-white hair agent, a hair dye agent, a treatment agent, an anti-aging agent, an antioxidant, a collagen synthesis promoter, an anti-wrinkle agent, an anti-acne agent, vitamin, an ultraviolet absorber, an aromatic, a coloring agent, an anhidrotic, a cooling agent, a warming agent, a melanin generation suppressant, a melanocyte activator, a cleansing agent, or a slimming agent; the functional food ingredient is vitamin, mineral, an antioxidant, an anti-stress agent, a nutritious supplement, amino acids, carotenoid, or a fruit or vegetable extract; and the pharmaceutical ingredient is a hair growth stimulant, a hair restorer, a hair growing agent, an antibiotic, an anti-cancer agent, an anti-inflammatory agent, an antiallergic agent, a hormone agent, an antithrombotic agent, an immunosuppressive agent, a therapeutic agent for skin disease, an antifungal agent, a nucleic acid agent, an anesthetic, an antipyretic, an analgesic, an antipruritic agent, an antihydropic, an antitussive expectorant, an antiepileptic, an antiparkinson agent, a sedative hypnotic, an antianxiety agent, an analeptic, an agent for psychoneurosis, a muscle relaxant, an antidepressant, a combination cold remedy, an autonomic agent, a spasmolytic agent, a sweater, an anhidrotic, a cardiac stimulant, a therapeutic agent for arrhythmia, an antiarrhythmic agent, an angiotonic, a vasodilator, a hypotensive agent, an antidiabetic agent, a therapeutic agent for hyperlipidemia, a respiratory stimulant, an antitussive agent, vitamin, a remedy for parasitic skin disease, a homeostatic regulator, polypeptide, hormone, a parakeratosis suppressant, vaccine, or a skin softener.

The present invention further provides a dispersed material, which comprises the casein particle according to the present invention in a dispersion medium.

The present invention further provides a method for producing the casein particle according to the present invention, which comprises dispersing casein in a solution having pH that is lower than an isoelectric point of the casein.

The present invention further provides a method for producing the casein particle according to claim 1, which comprises the following steps (a) and (b):

(a) a step of mixing casein into an acidic aqueous medium from pH 0.5 or more to less than pH 7; and
(b) a step of increasing the pH of the solution obtained in the step (a) above to a pH value that is ±pH 0.5 or more apart from the isoelectric point of the casein, while stirring the solution.

The present invention further provides a method for producing the casein particle according to claim 4, which comprises the following steps (a), (b) and (c):

(a) a step of mixing casein into an acidic aqueous medium from pH 0.5 or more to less than pH 7;
(b) a step of increasing the pH of the solution obtained in the step (a) above to a pH value that is ±pH 0.5 or more apart from the isoelectric point of the casein, while stirring the solution; and
(c) a step of adding at least one type of active substance to the solution obtained in the step (a) or (b) above.

According to the present invention, it is possible to provide a positively charged nanoparticle, which can be produced without using surfactants or synthetic polymers, the size of which can be controlled, which is stable at acidic condition, and which contains an active substance therein.

### PREFERRED EMBODIMENT OF THE INVENTION

Hereinafter, the present invention will be more specifically described.

The casein particle of the present invention is a particle characterized in that it has positive zeta potential. It preferably has a mean particle diameter from 10 nm or more to less than 300 nm, in terms of particle size.

In the present invention, a casein nanoparticle having a desired size can be produced. In addition, utilizing the interaction between a fat-soluble active substance and a casein hydrophobic portion, an active substance can be incorporated into the casein nanoparticle. Moreover, these particles are stably present in an aqueous solution. Such fat-soluble substance has a ClogP value of preferably greater than 0, more preferably 1 or greater, and further preferably 3 or greater. Moreover, by mixing casein with an ionic compound, an ionic polysaccharide, or a different type of ionic protein to prepare a mixed particle, the casein is able to contain an ionic active substance therein. That is to say, according to the present invention, a nanoparticle containing a highly safe active substance therein can be produced without using surfactants or synthetic polymers.

The casein nanoparticle of the present invention has a positive charge, and its zeta potential is positive. Zeta potential acts as an indicator in evaluation of the dispersion and/or aggregation properties of particles, interaction, and surface modification. The zeta potential can be measured by a known method. The zeta potential of the casein nanoparticle of the present invention is positive, and it is preferably 3 to 30 (mV).

The mean particle size of the casein nanoparticle of the present invention is usually 10 nm or more to less than 300 nm, preferably 10 to 100 nm, and more preferably 10 to 50 nm.

The casein nanoparticle of the present invention preferably comprises at least one active substance. The amount of the active substance is not particularly limited. The casein nanoparticle generally comprises an active substance in an amount 0.001 to 100 times greater than casein, in terms of the weight of the casein.

The casein used in the present invention is not particularly limited in origin and may be derived from either milk or beans. As such casein, α-casein, β-casein, γ-casein, κ-casein, and a mixture thereof can be used. A gene recombinant casein may also be used. The casein of the present invention can preferably be used in the form of casein sodium. The casein can be used singly or in combination of two or more types.

An example of the method for producing the casein nanoparticle of the present invention is a method comprising: (a) a step of mixing casein into an acidic aqueous medium from pH 0.5 or more to less than pH 7; and (b) a step of increasing the pH of the solution obtained in the step (a) above to a pH value that is ±pH 0.5 or more apart from the isoelectric point of the casein, while stirring the solution. The aforementioned step (b) can also be carried out by pouring the solution obtained in the step (a) above into an aqueous medium of pH 3.5 to 12.

When an active substance is incorporated into the particle or is retained on the particle surface, at least one type of active substance may be added to the solution obtained in the step (a) or (b) above.

The temperature of the acidic aqueous medium can be set as appropriate, and it can be normally 0° C. to 80° C., and preferably 25° C. to 70° C. Specific examples of such acidic aqueous medium used herein include aqueous solutions using organic acids such as citric acid, ascorbic acid, gluconic acid, carboxylic acid, tartaric acid, succinic acid, acetic acid, phthalic acid, trifluoroacetic acid, morpholinoethanesulfonic acid, 2-[4-(2-hydroxyethyl)-1-piperazinyl]ethanesulfonic acid, lactic acid, malonic acid, maleic acid, malic acid, glucuronic acid and mucic acid; and inorganic acids such as hydrochloric acid, perchloric acid and carbonic acid. The pH of such aqueous solution is pH 0.5 or more to less than pH 7. Since aggregation and precipitation occurs if the pH is close to the isoelectric point of casein, the pH is preferably pH 0.5 or more to less than pH 6, and more preferably pH 0.5 or more to pH 4 or less. As acid, organic acids are preferably used. More preferably, citric acid, malic acid, and tartaric acid are used. In the present invention, the temperature at which casein is mixed into an acidic aqueous medium of pH 0.5 or more is preferably 0° C. to 80° C., and more preferably 10° C. to 60° C. It is further preferably 20° C. to 40° C.

As a method of increasing the pH of an acidic aqueous medium solution while stirring, after casein has been mixed into the solution, the addition of alkali dropwise to the solution is preferable because it is simple. However, the type of such method is not particularly limited, as long as it satisfies solubility, a temperature, and a stirring state.

The stirring speed can be determined as appropriate. It can be set at normally 100 rpm to 3,000 rpm, and preferably 200 rpm to 2,000 rpm.

Examples of alkali added dropwise to the solution include, but are not limited to, aqueous solutions using inorganic bases such as sodium phosphate, potassium phosphate, calcium hydroxide, sodium hydroxide, potassium hydroxide and magnesium hydroxide, and aqueous solutions using organic bases such as tri(hydroxymethyl)aminomethane and ammonia. Inorganic bases are preferable. Sodium hydroxide and potassium hydroxide are more preferable.

In such method of mixing casein into an acidic aqueous medium solution and then increasing the pH of the solution while stirring, the preferred pH after the increase in the temperature is a pH value that is ±pH 0.5 or more, preferably ±pH 4 or less, and more preferably ±pH 3 or less, apart from the isoelectric point of the casein.

The method comprising mixing casein into an acidic aqueous medium solution and injecting the resulting solution into an aqueous medium of pH 3.5 to 12 is preferably carried out with the use of a syringe because of the simplicity of its operation. However, the method is not particularly limited, as long as it satisfies an injection rate, solubility, a temperature, and a stirring state. In general, the solution can be injected at an injection rate of 1 mL/min to 100 mL/min. The temperature of the acidic aqueous medium can appropriately be determined. The temperature can be determined generally at 0° C. to 80° C., and preferably at 25° C. to 70° C. The temperature of the aqueous medium can appropriately be determined. The temperature can be determined generally at 0° C. to 80° C., and preferably at 25° C. to 60° C. A stirring speed can be set appropriately and can be normally 100 rpm to 3000 rpm, and preferably 200 rpm to 2000 rpm.

An aqueous solution or buffer solution of an organic acid or base, or an inorganic acid or base, can be used as an aqueous medium in the present invention.

Specific examples of such aqueous medium include, but are not limited to, aqueous solutions using organic acids such as citric acid, ascorbic acid, gluconic acid, carboxylic acid, tartaric acid, succinic acid, acetic acid, phthalic acid, trifluoroacetic acid, morpholinoethanesulfonic acid, 2-[4-(2-hydroxyethyl)-1-piperazinyl]ethanesulfonic acid, malonic acid, maleic acid, malic acid, glucuronic acid, and mucic acid; organic bases such as tris(hydroxymethyl), aminomethane, and ammonia; inorganic acids such as hydrochloric acid, perchloric acid, and carbonic acid; and inorganic bases such as sodium phosphate, potassium phosphate, calcium hydroxide, sodium hydroxide, potassium hydroxide, and magnesium hydroxide.

The concentration of the aqueous medium used in the present invention is preferably approximately 5 mM to approximately 500 mM, and more preferably approximately 5 mM to approximately 200 mM.

The casein particle of the present invention preferably comprises an active substance in a particle thereof and/or on a particle surface thereof. The type of the active substance used in the present invention can be selected from among a cosmetic ingredient, a functional food ingredient, and a pharmaceutical ingredient, for example. The casein nanoparticle of the present invention is characterized in that an anionic substance can be incorporated therein or can be maintained on the surface. Accordingly, among the below-mentioned ingredients, anionic compounds are more preferable.

Examples of a cosmetic ingredient include a moisturizer, a skin-lightening agent, a hair growth stimulant, a hair restorer, a hair growing agent, an anti-white hair agent, an anti-aging agent, an antioxidant, a collagen synthesis promoter, an anti-wrinkle agent, an anti-acne agent, vitamin, an ultraviolet absorber, an aromatic, a coloring agent, an anhidrotic, a cooling agent, a warming agent, a melanin generation suppressant, a melanocyte activator, a cleansing agent, and a slimming agent.

Examples of a functional food ingredient include vitamin, mineral, an antioxidant, an anti-stress agent, a nutritious supplement, amino acids, carotenoid, and fruit and vegetable extracts.

Examples of a pharmaceutical ingredient include a hair growth stimulant, a hair restorer, a hair growing agent, an antibiotic, an anti-cancer agent, an anti-inflammatory agent, an antiallergic agent, a hormone agent, an antithrombotic agent, an immunosuppressive agent, a therapeutic agent for skin disease, an antifungal agent, a nucleic acid agent, an anesthetic, an antipyretic, an analgesic, an antipruritic agent, an antihydropic, an antitussive expectorant, an antiepileptic, an antiparkinson agent, a sedative hypnotic, an antianxiety agent, an analeptic, an agent for psychoneurosis, a muscle relaxant, an antidepressant, a combination cold remedy, an autonomic agent, a spasmolytic agent, a sweater, an anhidrotic, a cardiac stimulant, a therapeutic agent for arrhythmia, an antiarrhythmic agent, an angiotonic, a vasodilator, a hypotensive agent, an antidiabetic agent, a therapeutic agent for hyperlipidemia, a respiratory stimulant, an antitussive agent, vitamin, a remedy for parasitic skin disease, a homeostatic regulator, polypeptide, hormone, a parakeratosis suppressant, vaccine, and a skin softener. The aforementioned active substance can be used alone or in combination of two or more types.

Specific examples of the moisturizer used in the present invention are listed below. However, in the present invention, the moisturizer is not limited to these compounds. It includes hyaluronic acid, ceramide, Lipidure, isoflavone, amino acids, and collagen. The aforementioned moisturizer can be used alone or in combination of two or more types.

Specific examples of the skin-lightening agent used in the present invention are listed below. However, in the present invention, the skin-lightening agent is not limited to these compounds. It includes vitamin C derivatives, hydroquinones, arbutin, Rucinol, and ellagic acid. The aforementioned skin-lightening agent can be used alone or in combination of two or more types.

Specific examples of the anti-aging agent and the antioxidant used in the present invention are listed below. However, in the present invention, the anti-aging agent and the antioxidant are not limited to these compounds. It includes carotenes, retinoic acid, retinol, vitamin C derivatives, kinetin, astaxanthin, tretinoin, vitamin E and a derivative thereof, sesamin, α-lipoic acid, coenzyme Q10, and flavonoids. The aforementioned anti-aging agent and antioxidant can be used alone or in combination of two or more types.

Specific examples of the anti-acne agent used in the present invention are listed below. However, in the present invention, the anti-acne agent is not limited to these compounds. It includes salicylic acid, resorcin, retinoic acid, nadifloxacin, an aminoglycoside antibiotic, a tetracycline antibiotic, and a lincomycin antibiotic. The aforementioned anti-acne agent can be used alone or in combination of two or more types.

Specific examples of an anticancer agent used in the present invention are listed below. However, in the present invention, the anticancer agent is not limited to these compounds. It includes, but are not limited to, fluorinated pyrimidine-based antimetabolites (e.g., 5-fluorouracil (5FU), tegafur, doxifluridine, and capecitabine); antibiotics (e.g., mitomycin (MMC) and Adriacin (DXR)); purine antimetabolites (e.g., folic acid antimetabolites (such as methotrexate) and mercaptopurine); active metabolites of vitamin A (e.g., antimetabolites (such as hydroxycarbamide), tretinoin, and tamibarotene); molecular target drugs (e.g., Herceptin and imatinib mesilate); platinum preparations (e.g., Briplatin or Randa (CDDP), Paraplatin (CBDC), Elplat (Oxa), and Aqupla); plant alkaloid drugs (e.g., Topotecin or Campto (CPT), taxol (PTX), Taxotere (DTX), and etoposide); alkylating agents (e.g., busulfan, cyclophosphamide, and ifomide); anti-androgen drugs (e.g., bicalutamide and flutamide); estrogen drugs (e.g., fosfestrol, chlormadinone acetate, and estramustine phosphate); LH-RH drugs (e.g., Leuplin and Zoladex); anti-estrogen drugs (e.g., tamoxifen citrate and toremifene citrate); aromatase inhibitors (e.g., fadrozole hydrochloride, anastrozole, and exemestane); corpus luteum hormone drugs (e.g., medroxyprogesterone acetate); and BCG. The aforementioned anticancer agent can be used alone or in combination of two or more types.

Specific examples of the antiallergic agent used in the present invention are listed below. However, in the present invention, the antiallergic agent is not limited to these compounds. It includes: mediator release inhibitors such as sodium cromoglycate and tranilast; histamine H1 antagonists such as ketotifen fumarate and azelastine hydrochloride; thromboxane inhibitors such as ozagrel hydrochloride; leukotriene antagonists such as pranlukast; and suplatast tosilate. The aforementioned antiallergic agent can be used alone or in combination of two or more types.

Specific examples of the immunosuppressive agent used in the present invention are listed below. However, in the present invention, the immunosuppressive agent is not limited to these compounds. It includes rapamycin, tacrolimus, cyclosporine, prednisolone, methylprednisolone, mycophenolate mofetil, azathioprine, and mizoribine. The aforementioned immunosuppressive agent can be used alone or in combination of two or more types.

The type of the hair growing ingredient used in the present invention is not particularly limited. Such hair growing ingredient can be selected from among cosmetic ingredients and pharmaceutical ingredients, for example. Specific examples of the hair growing ingredient contained in the protein nanoparticle of the present invention include: glycyrrhetic acid or a derivative thereof; glycyrrhizinic acid or a derivative thereof; hinokitiol; vitamin E or a derivative thereof; a vitamin C derivative; 6-benzylaminopurine; nicotinic acid amide; benzyl nicotinate; tocopherol nicotinate; nicotinic acid β-butoxy ester; isopropylmethylphenol; pentadecanoic acid or a derivative thereof; cepharanthin; finasteride; t-flavanone; an antioxidant such as carotenoid or kinetin; ethinyl estradiol; pantothenyl alcohol; pantothenyl ethyl ether; minoxidil or an analogue thereof; carpronium chloride; and adenosine. The aforementioned hair growing ingredient can be used alone or in combination of two or more types.

In the present invention, an ionic compound and an additive interacting with the active substance may be used. The aforementioned ionic compound and additive each preferably have a charge opposite to the charge of the active substance. The ionic compound and additive are each preferably added in 0.1 to 10 equivalent amounts with respect to the molecular weight of the active substance.

The ionic compound and additive interacting with the active substance may be either cationic or anionic compounds. The compound preferably has a hydrophobic portion and a hydrophilic portion in a molecule thereof. The hydrophobic portion preferably has one or more of a conjugate structure, a cyclic structure, a long-chain alkyl structure, and a long-chain alkylene structure. More preferably, it has a steroid structure, or a benzoyl, biphenyl or phenyl structure.

Specific examples of the aforementioned ionic compound and additive interacting with the active substance will be given below. However, the ionic compound and additive are not limited thereto. Particularly, anionic compounds will be listed below: lauryl sulfate and a salt thereof, myristyl sulfate and a salt thereof, cetyl sulfate and a salt thereof, lauryl phosphate and a salt thereof, toluenesulfonic acid and a salt thereof, toluic acid and a salt thereof; dimethylbenzoic acid and a salt thereof, acetylbenzenesulfonic acid and a salt thereof, benzoic acid and a salt thereof, biphenylcarboxylic acid and a salt thereof, benzoylbenzoic acid and a salt thereof, cholic acid and a salt thereof, glycocholic acid and a salt thereof, taurocholic acid and a salt thereof, chenodeoxycholic acid and a salt thereof, hyocholic acid and a salt thereof, ursodeoxycholic acid and a salt thereof, cyprinol and a salt thereof, deoxycholic acid and a salt thereof, lithocholic acid and a salt thereof, hyodeoxycholic acid and a salt thereof, glycyrrhetinic acid and a salt thereof, and formic acid cholesterol and a salt thereof.

In the present specification, in a case in which at least one active substance is added to the solution obtained in the aforementioned step (a) or (b) above, a solution prepared by dissolving the active substance in water or an organic solvent miscible at least 10% by weight with water may be added. Specific examples of an organic solvent miscible at least at 10% by weight with water used in the present invention are listed below. However, in the present invention, the organic solvent is not limited to these compounds. Preferred examples include higher alcohols and polyhydric alcohols such as ethanol, isopropanol, propylene glycol, butylene glycol, ethylene glycol and glycerin, and water-soluble organic solvents such as acetone and THF.

In the present invention, the active substance can be added in the form of an aqueous liposome dispersion containing the active substance therein. Specific examples of lipid for forming the liposome used in the present invention are listed below. However, in the present invention, the lipid is not limited to these compounds. It includes egg-yolk lecithin, soybean lecithin, egg-yolk phosphatidylcholine, dipalmitoylphosphatidylcholine, and dimyristoylphosphatidylcholine. The liposome may comprise phosphatidylserines, phosphatidylethanolamines, and cholesterol, in addition to the compounds described above.

To the casein particle of the present invention, one or more ingredients selected from among cyclodextrin, lipid, a different type of protein, a cationic or anionic polysaccharide, and a cationic or anionic protein can be added.

Specific examples of cyclodextrin used in the present invention are listed below. However, in the present invention, the cyclodextrin is not limited to these compounds. It includes α-cyclodextrin, β-cyclodextrin, γ-cyclodextrin, 2,6-di-O-methyl-α-cyclodextrin, 2,6-di-O-methyl-β-cyclodextrin, glucuronyl glucosyl-β-cyclodextrin, heptakis(2,6-di-O-methyl)-β-cyclodextrin, 2-hydroxyethyl-β-cyclodextrin, hydroxypropyl-β-cyclodextrin, 6-O-α-maltosyl-α-cyclodextrin, methyl-β-cyclodextrin, 2,3,6-tri-O-methyl-β-cyclodextrin, and 6-O-α-D-glucosyl-α-cyclodextrin.

Specific examples of lipid used in the present invention are listed below. However, in the present invention, the lipid is not limited to these compounds. It includes phosphatidylcholine (lecithin), phosphatidylethanolamine, phosphatidylserine, phosphatidylinositol, phosphatidylglycerol, diphosphatidylglycerol, sphingosines, ceramide, oleic acid, linoleic acid, linolenic acid, palmitic acid, myristic acid, stearic acid, soybean oil, olive oil, and squalane.

A different type of protein used in the present invention is not particularly limited in kind. Preferably, a protein with a molecular weight of approximately 10,000 to 1,000,000 is used. The protein is not particularly limited in origin. Preferably, a protein derived from a human is used. Specific examples of the protein are listed below. However, in the present invention, the protein is not limited to these compounds. Examples of such a different type of protein that can be used herein include collagen, gelatin, albumin, transferrin, fibrin, fibrinogen, globulin, fibroin, laminin, fibronectin, and vitronectin. Moreover, the origin of the protein is not particularly limited, and any one of bovine, swine, fish and a gene recombinant protein can be used. Among them, gelatin and albumin are preferable.

An anionic polysaccharide used in the present invention is a polysaccharide having an acidic polar group such as a carboxyl group, sulfuric acid group, or phosphoric acid group. Specific examples thereof are listed below. However, in the present invention, the anionic polysaccharide is not limited to these compounds. It includes chondroitin sulfate, dextran sulfate, carboxymethyldextran, alginic acid, pectin, carrageenan, fucoidan, agaropectin, porphyran, karaya gum, gellan gum, xanthan gum, and hyaluronic acids.

A cationic polysaccharide used in the present invention is a polysaccharide having a basic polar group such as an amino group. Specific examples thereof are listed below. However, in the present invention, the cationic polysaccharide is not limited to these compounds. It includes those comprising glucosamine (e.g., chitin and chitosan) or galactosamine as a constituent monosaccharide.

An anionic protein used in the present invention includes a protein and a lipoprotein with an isoelectric point more basic than the physiological pH. Specific examples thereof are listed below. However, in the present invention, the anionic protein is not limited to these compounds. It includes polyglutamic acid, polyaspartic acid, cytochrome C, ribonuclease, trypsinogen, chymotrypsinogen, and α-chymotrypsin.

A cationic protein used in the present invention includes a protein and a lipoprotein with an isoelectric point more acidic than the physiological pH. Specific examples thereof are listed below. However, in the present invention, the cationic protein is not limited to these compounds. It includes polylysine, polyarginine, histone, protamine, and ovalbumin.

The casein nanoparticle of the present invention preferably comprises the active substance therein. Such a casein nanoparticle comprising the active substance can be administered to the affected part for use. Specifically, the casein nanoparticle of the present invention is useful as a drug delivery agent.

In the present invention, the usage of the drug delivery agent is not particularly limited. For example, the drug delivery agent is used as a transdermal agent, a local therapeutic agent, an oral therapeutic agent, a cosmetic product, a supplement, and the like.

In the present invention, the drug delivery agent preferably comprises 0.01% to 50% by weight of the protein nanoparticle, and more preferably comprises 0.1% to 10% by weight of the protein nanoparticle. The amount of ethanol contained in the drug delivery agent is preferably 20% or less, and more preferably 10% or less, with respect to the total amount.

In the present invention, the drug delivery agent may comprise an additive. The type of such additive is not particularly limited. Examples of such additive include a moisturizer, a softener, a percutaneous absorption promoter, an antiseptic, a coloring agent, an aromatic, and a pH adjuster.

Specific examples of the moisturizer that can be used in the present invention include, but are not limited to, agar, diglycerin, distearyldimonium hectorite, butylene glycol, polyethylene glycol, propylene glycol, sodium hyaluronate, hexylene glycol, coix seed extract, and vaserine.

Specific examples of the softener that can be used in the present invention include, but are not limited to, glycerin, mineral oil, and emollient ingredients (e.g. isopropyl isostearate, polyglyceryl isostearate, isotridecyl isononanoate, octyl isononanoate, oleic acid, glyceryl oleate, cacao butter, cholesterol, mixed fatty acid triglyceride, dioctyl succinate, sucrose acetate stearate, cyclopentanesiloxane, sucrose distearate, octyl palmitate, octyl hydroxystearate, arachidyl behenate, sucrose polybehenate, polymethylsilsesquioxane, myristyl alcohol, cetyl myristate, myristyl myristate, and hexyl laurate).

Specific examples of the percutaneous absorption promoter that can be used in the present invention include, but are not limited to, ethanol, isopropyl myristate, citric acid, squalane, oleic acid, menthol, N-methyl-2-pyrrolidone, diethyl adipate, diisopropyl adipate, diethyl sebacate, diisopropyl sebacate, isopropyl palmitate, isopropyl oleate, octyldodecyl oleate, isostearyl alcohol, 2-octyldodecanol, urea, vegetable oil, and animal oil.

Specific examples of the antiseptic that can be used in the present invention include, but are not limited to, benzoic acid, sodium benzoate, ethylparaben, potassium sorbate, sodium sorbate, sorbic acid, sodium dehydroacetate, and methylparaben.

Specific examples of the coloring agent that can be used in the present invention include, but are not limited to, kaoline, carmine, ultramarine blue, chromium oxide, and iron oxide.

Specific examples of the aromatic that can be used in the present invention include, but are not limited to, musk, acacia oil, anise oil, ylang-ylang oil, cinnamon oil, jasmine oil, sweet orange oil, spearmint oil, geranium oil, thyme oil, neroli oil, mint oil, Japanese cypress oil, fennel oil, peppermint oil, bergamot oil, lime oil, lavender oil, lemon oil, lemongrass oil, rose oil, rosewood oil, anisaldehyde, geraniol, citral, civetone, muscone, limonene, and vanillin.

Specific examples of the pH adjuster that can be used in the present invention include, but are not limited to, sodium citrate, sodium acetate, sodium hydroxide, potassium hydroxide, and phosphoric acid.

Preferred methods of administering the casein nanoparticle of the present invention include transdermal and transmucosal absorption. Specific examples of such administration method that can be applied in the present invention include, but are not limited to, an external liquid preparation, a poultice, an embrocation, a cleaning agent, a bath preparation, a disinfectant, an ointment, a gel, a cream, a paste, a cataplasm, a plaster, a wound surface-coating agent, a wound surface-coating gauze, a hemostatic, an adhesive, an adhesive tape, a percutaneous-absorption-type adhesive tape, a wound surface protecting agent, an aerosol, a lotion, a tonic, a liniment, an emulsion, a suspension, a saturant, a tincture, a powder, a foam, a cosmetic lotion, a massage cream, a nourishing cream, a pack, a sheet-form external skin preparation, a skin-adhesive-type cosmetic product, a lipstick, a makeup base, a foundation, a shampoo, a conditioner, a body soap, a soap, a bath form, a transnail agent, a nasal mucosal agent, an oral mucosal agent, a rectal mucosal agent, a vaginal mucosal agent, an eye mucosal agent, and a lung mucosal agent.

The dose of the casein nanoparticle of the present invention can be set appropriately according to the body weight of a patient, the state of the disease, and so on. In general, approximately 10 μg to 100 mg/kg can be administered per administration. Preferably, approximately 20 μg to 50 mg/kg can be administered per administration.

The present invention will be described more specifically in the following examples. However, these examples are not intended to limit the scope of the present invention.

### EXAMPLES

### Example 1

100 mg of casein (derived from milk; manufactured by Wako Pure Chemical Industries, Ltd.; isoelectric point: 4.3) was mixed into 10 ml of 50 mM citric acid solution (pH 1.9). NaOH was added to the mixed solution, so that the pH could be stably maintained at pH 3. The mean particle diameter of the aforementioned particles was measured using a light scattering photometer (Nano-ZS; manufactured by Malvern Instruments Ltd.). As a result, it was found to be 22 nm, and the zeta potential was found to be 16 mV (Table 1). In addition, it was confirmed that the particles had been stably dispersed at 4° C. for 10 days.

### Examples 2 and 3

The casein nanoparticles were produced in the same manner as that of Example 1 with the exception that the final pH after addition of NaOH was set at pH 2.1 (Example 2) or pH 3.9 (Example 3). Thereafter, the particle size (nm) and the zeta potential (mV) were measured. The results are shown in Table 1.

### Comparative Examples 1 to 5

The casein nanoparticles were produced in the same manner as that of Example 1 with the exception that the final pH after addition of NaOH was set at pH 5.0 (Comparative Example 1), pH 6.5 (Comparative Example 2), pH 7.0 (Comparative Example 3), pH 7.9 (Comparative Example 4), or pH 10.0 (Comparative Example 5). Thereafter, the particle size (nm) and the zeta potential (mV) were measured. The results are shown in Table 1.

TABLE 1

| | | | | Compara. | Compara | Compara. | Compara. | Compara. |
|---|---|---|---|---|---|---|---|---|
| | Example 2 | Example 1 | Example 3 | Example 1 | Example 2 | Example 3 | Example 4 | Example 5 |
| pH | 2.1 | 3.0 | 3.9 | 5.0 | 6.5 | 7.0 | 7.9 | 10.0 |
| Particle size | 46 | 22 | 1110 | 184 | 22 | 21 | 19 | 18 |
| (nm) | | | | | | | | |
| Zeta Potential | 23 | 16 | 4 | −15 | −14 | −15 | −15 | −15 |
| (mV) | | | | | | | | |
| Remarks | Present | Present | Present | Compara. | Compara. | Compara. | Compara. | Compara. |
| | Invention | Invention | Invention | Example. | Example | Example. | Example. | Example. |

In Comparative Examples 1 to 5 shown in Table 1, particles had negative zeta potential, and thus the object of the present invention could not be achieved. Although the pH of the particles of Comparative Examples 1 to 5 was then converted to pH 3, they were not re-dispersed. Accordingly, it became clear that, when the cationic property of casein nanoparticles is utilized, the nanoparticles need to be produced in the acidic range. In Example 3, aggregation and/or precipitation occurred.

### Example 4

Casein nanoparticles were produced using malic acid, instead of the citric acid of Example 1, and a dispersion solution of pH 2.4 was obtained. The mean particle diameter of the aforementioned particles was measured using a light scattering photometer (Nano-ZS; manufactured by Malvern Instruments Ltd.). As a result, it was found to be 38 nm, and the zeta potential was found to be 24 mV. In addition, it was confirmed that the particles had been stably dispersed at 4° C. for 10 days.

### Example 5

Casein nanoparticles were produced using tartaric acid, instead of the citric acid of Example 1, and a dispersion solution of pH 2.4 was obtained. The mean particle diameter of the aforementioned particles was measured using a light scattering photometer (Nano-ZS; manufactured by Malvern Instruments Ltd.). As a result, it was found to be 11 nm, and the zeta potential was found to be 23 mV. In addition, it was confirmed that the particles had been stably dispersed at 4° C. for 10 days.

### Example 6

Casein nanoparticles were produced in the same way as in Example 1, except that the amount of casein used in Example 1 was changed to 200 mg. A dispersion solution of pH 2.6 was obtained. The mean particle diameter of the aforementioned particles was measured using a light scattering photometer (Nano-ZS; manufactured by Malvern Instruments Ltd.). As a result, it was found to be 26 nm, and the zeta potential was found to be 18 mV.

### Comparative Example 6

100 mg of casein (derived from milk; manufactured by Wako Pure Chemical Industries, Ltd.) was mixed into 10 mL of 50 mM phosphate buffer (pH 10). While stirring, hydrochloric acid was added to the mixed solution, so that the pH was adjusted to pH 4. As a result, the solution became clouded and aggregated.

### Comparative Example 7

100 mg of casein (derived from milk; manufactured by Wako Pure Chemical Industries, Ltd.) was mixed into 10 mL of 50 mM phosphate buffer (pH 10). While stirring, hydrochloric acid was added to the mixed solution, so that the pH was adjusted to pH 2. As a result, the solution became clouded and aggregated.

1. A casein particle, wherein zeta potential is positive.

2. The casein particle according to claim 1, which has a mean particle diameter from 10 nm or more to less than 300 nm, in terms of particle size.

3. A casein particle having a mean particle diameter from 10 nm or more to less than 300 nm, which is produced by the following steps (a) and (b):
(a) a step of mixing casein into an acidic aqueous medium from pH 0.5 or more to less than pH 7; and
(b) a step of increasing the pH of the solution obtained in the step (a) above to a pH value that is ±pH 0.5 or more apart from the isoelectric point of the casein, while stirring the solution.

4. The casein particle according to claim 1, which comprises an active substance in the particle thereof and/or on the particle surface thereof.

5. The casein particle according to claim 4, wherein the active substance is an ionic substance or a fat-soluble substance.

6. The casein particle according to claim 4, wherein the active substance is an anionic substance.

7. The casein particle according to claim 4, which is produced by the following steps (a), (b), and (c):
(a) a step of mixing casein into an acidic aqueous medium from pH 0.5 or more to less than pH 7;
(b) a step of increasing the pH of the solution obtained in the step (a) above to a pH value that is ±pH 0.5 or more apart from the isoelectric point of the casein, while stirring the solution; and
(c) a step of adding at least one type of active substance to the solution obtained in the step (a) or (b) above.

8. The casein particle according to claim 4, wherein the active substance is a cosmetic ingredient, a functional food ingredient, or a pharmaceutical ingredient.

9. The casein particle according to claim 8, wherein the cosmetic ingredient is a moisturizer, a skin-lightening agent, a hair growth stimulant, a hair restorer, a hair growing agent, an anti-white hair agent, a hair dye agent, a treatment agent, an anti-aging agent, an antioxidant, a collagen synthesis promoter, an anti-wrinkle agent, an anti-acne agent, vitamin, an ultraviolet absorber, an aromatic, a coloring agent, an anhidrotic, a cooling agent, a warming agent, a melanin generation suppressant, a melanocyte activator, a cleansing agent, or a slimming agent; the functional food ingredient is vitamin, mineral, an antioxidant, an anti-stress agent, a nutritious supplement, amino acids, carotenoid, or a fruit or vegetable extract; and the pharmaceutical ingredient is a hair growth stimulant, a hair restorer, a hair growing agent, an antibiotic, an anti-cancer agent, an anti-inflammatory agent, an antiallergic agent, a hormone agent, an antithrombotic agent, an immunosuppressive agent, a therapeutic agent for skin disease, an antifungal agent, a nucleic acid agent, an anesthetic, an antipyretic, an analgesic, an antipruritic agent, an antihydropic, an antitussive expectorant, an antiepileptic, an antiparkinson agent, a sedative hypnotic, an antianxiety agent, an analeptic, an agent for psychoneurosis, a muscle relaxant, an antidepressant, a combination cold remedy, an autonomic agent, a spasmolytic agent, a sweater, an anhidrotic, a cardiac stimulant, a therapeutic agent for arrhythmia, an antiarrhythmic agent, an angiotonic, a vasodilator, a hypotensive agent, an antidiabetic agent, a therapeutic agent for hyperlipidemia, a respiratory stimulant, an antitussive agent, vitamin, a remedy for parasitic skin disease, a homeostatic regulator, polypeptide, hormone, a parakeratosis suppressant, vaccine, or a skin softener.

10. A dispersed material, which comprises the casein particle according to claim 1 in a dispersion medium.

11. A method for producing the casein particle according to claim 1, which comprises dispersing casein in a solution having pH that is lower than an isoelectric point of the casein.

12. A method for producing the casein particle according to claim 1, which comprises the following steps (a) and (b):
(a) a step of mixing casein into an acidic aqueous medium from pH 0.5 or more to less than pH 7; and
(b) a step of increasing the pH of the solution obtained in the step (a) above to a pH value that is ±pH 0.5 or more apart from the isoelectric point of the casein, while stirring the solution.

13. A method for producing the casein particle according to claim 4, which comprises the following steps (a), (b) and (c):
(a) a step of mixing casein into an acidic aqueous medium from pH 0.5 or more to less than pH 7;
(b) a step of increasing the pH of the solution obtained in the step (a) above to a pH value that is ±pH 0.5 or more apart from the isoelectric point of the casein, while stirring the solution; and
(c) a step of adding at least one type of active substance to the solution obtained in the step (a) or (b) above.
